(19) United States
(12) Patent Application Publication
(10) Pub. No.: US 2016/0331866 A1
(43) Pub. Date: Nov. 17, 2016

(54) TISSUE REGENERATION MEMBRANE FOR REPAIR AND REGENERATION OF PERIPHERAL NERVE

(71) Applicants: NATIONAL CHENG KUNG UNIVERSITY, TAINAN CITY (TW); NATIONAL CHENG KUNG UNIVERSITY HOSPITAL, TAINAN CITY (TW)

(72) Inventors: Chia-Ching Wu, Tainan City (TW); Yuan-Yu Hsueh, Tainan City (TW); Duo-Hsiang Wang, Tainan City (TW)

(21) Appl. No.: 15/151,668

(22) Filed: May 11, 2016

(30) Foreign Application Priority Data
May 14, 2015 (TW) 104115422

(51) Int. Cl. A61L 27/24; A61K 31/616; A61K 31/381; A61L 27/54

(52) U.S. Cl. CPC A61L 27/24; A61L 27/54; A61K 31/616; A61K 31/381; A61L 2430/32; A61L 2300/41

(57) ABSTRACT

The present invention relates to a tissue regeneration membrane for repair and regeneration of peripheral nerve, which includes a collagen membrane and an anti-inflammatory medicinal composition dispersed therein uniformly, for wrapping an injured peripheral nerve. The collagen membrane protects the injured peripheral nerve from the infiltration of inflammatory cells, and the anti-inflammatory medicinal composition can inhibit neuroinflammation of the injured peripheral nerve, thereby promoting the repair and regeneration of the injured peripheral nerve.

### RELATED APPLICATIONS

This application claims priority to Taiwan Application Serial Number 104115422, filed May 14, 2015, which is herein incorporated by reference.

### BACKGROUND

1. Field of Invention

The present invention relates to a tissue regeneration membrane. More particularly, the present invention relates to a tissue regeneration membrane for repair and regeneration of peripheral nerve, thereby preventing the infiltration of inflammatory cells and inhibiting the nerve inflammation.

2. Description of Related Art

The recovery probability of peripheral nerve system (PNS) injuries in the youth is approximately 50% to 70%; however, the recovery probability of the PNS injuries in the elderly is decreasing, and the elderly may be not capable to recover completely. The incomplete healing of injured PNS often causes some complications such as neuropathic pain or complex regional pain syndrome (CRPS). Patients suffer massive pain, followed by functional loss and higher medical expense. The CRPS may be caused by acute or chronic injuries of PNS, for example, physical crush injury or compressive neuropathy. As peripheral nerves regenerate, excessive tissue adhesions and scars gives rise to the stimulation, suppress and pain of peripheral nerves. Nerve wrap (or called tissue regeneration membrane) can protect the injured nerves, prevent the formation of soft tissue adhesions and scars and promote the nerve regeneration.

Currently, some products of nerve wraps are commercially available, but there is no enough scientific evidence on their efficacy, and mechanisms involved in the therapy are uncertain. For example, NeuroWrap™ or NeuroMend™, both of which are wraps of bioabsorbable collagen fibers, claiming to protect injured nerves and to improve nerve recovery by minimizing nerve entrapment. However, those commercial products have not mentioned that the efficacy on how to facilitate nerve regeneration and prevent scar hypertrophy of nerves.

There is, however, a growing need for a tissue regeneration membrane for repair and regeneration of peripheral nerve, thereby improving the problems of the infiltration of inflammatory cells and the neuroinflammation surrounding the injured peripheral nerve.

### SUMMARY

Accordingly, the invention provides a tissue regeneration membrane for repair and regeneration of peripheral nerve, which includes uncrosslinked collagen, a difference defined between an average thickness of the tissue regeneration membrane and a diameter of the injured peripheral nerve is no more than 20 percents, for wrapping injured peripheral nerves and preventing the infiltration of peripheral inflammatory cells.

Moreover, the present invention provides a tissue regeneration membrane for repair and regeneration of peripheral nerve, in which the tissue regeneration membrane includes a collagen membrane and an anti-inflammatory medicinal composition dispersed therein uniformly, the collagen membrane is consisted of uncrosslinked collagen, and the collagen membrane can wrap an injured peripheral nerve, for inhibiting overinflammation inside and outside the injured nerve tissues, thereby promoting the recovery and the regeneration of injured peripheral nerve.

Furthermore, the present invention provides a tissue regeneration membrane for repair and regeneration of peripheral nerve, which includes a collagen membrane having an average thickness of 0.2 μm to 25 μm, and an anti-inflammatory medicinal composition dispersed in the collagen membrane uniformly, in which the collagen membrane is consisted of uncrosslinked collagen, and a difference defined between an average thickness of the tissue regeneration membrane and a diameter of the injured peripheral nerve is no more than 20 percents.

According to the aforementioned aspect, the invention provides a tissue regeneration membrane, which is consisted of uncrosslinked collagen. In an embodiment, the tissue regeneration membrane has an average thickness of 0.2 μm to 25 μm, for wrapping an injured peripheral nerve with a desired injured length. The aforementioned injured peripheral nerve has a larger diameter and a smaller diameter in the desired injured length. A first difference is defined between the larger diameter and the average thickness, and a second difference is defined between the average thickness and the smaller diameter. The first difference is no more than 20 percents based on the larger diameter as 100 percents, or the second difference is no more than 20 percents based on the smaller diameter as 100 percents.

According to the aforementioned aspect, the present invention further provides a tissue regeneration membrane for repair and regeneration of peripheral nerve, in which the tissue regeneration membrane includes a collagen membrane and an anti-inflammatory medicinal composition dispersed therein uniformly. In an embodiment, the tissue regeneration membrane has an average thickness of 0.2 μm to 25 μm. In another embodiment, a first difference is defined between the larger diameter and the average thickness, and a second difference is defined between the average thickness and the smaller diameter. The first difference can be exemplified as no more than 20 percents based on the larger diameter as 100 percents, or the second difference can be exemplified as no more than 20 percents based on the smaller diameter as 100 percents.

According to an embodiment, the aforementioned collagen membrane can be consisted of uncrosslinked collagen type I.

According to an embodiment, the aforementioned first difference is 5 percents to 20 percents based on the larger diameter as 100 percents, and preferably 5 percents to 10 percents. In another example, the aforementioned second difference is 5 percents to 20 percents based on the smaller diameter as 100 percents, and preferably 5 percents to 10 percents.

According to an embodiment, the aforementioned tissue regeneration membrane can have a larger thickness and a smaller thickness, a third difference is defined between the larger thickness and the smaller thickness, and the third difference can be no more than 5 μm, preferably no more than 2 μm, and more preferably no more than 500 nm.

According to an embodiment, the aforementioned desired injured length can include but be not limited to 5 mm to 50 mm.

According to an embodiment, the aforementioned anti-inflammatory medicinal composition can further include an anti-inflammatory active ingredient and a medicinally acceptable carrier. In an example, the anti-inflammatory active ingredient can include but be not limited to at least one of cyclooxygenase inhibitor, 5-lipoxygenase (5-LO) inhibitor and leukotriene receptor antagonist. In an example, examples of the aforementioned anti-inflammatory active ingredient can be acetylsalicylic acid, Indomethacin, Zileuton, Montelukast or any combination thereof, for examples. In another example, the aforementioned anti-inflammatory active ingredient can be used alone or in combination of two or more.

According to an embodiment, an amount of the aforementioned anti-inflammatory active ingredient in the wrapped peripheral nerve tissue can be 20 ng/mL to 500 ng/mL, for example.

With application to the tissue regeneration membrane for repair and regeneration of the peripheral nerve, it includes a collagen membrane having an average thickness very close to a diameter of the injured peripheral nerve and an anti-inflammatory medicinal composition dispersed the collagen membrane uniformly, for wrapping and facilitating regeneration of the injured peripheral nerves.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIGS. 1A to 1D are partial flow diagrams depicting the establishment of an animal model in a surgical manipulation process according to one embodiment of the present invention;

FIGS. 2A to 2F are haematoxylin- and eosin-stained histological images of the peripheral nerve tissues according to the second embodiment of the present invention;

FIGS. 3A to 3F are glial fibrillary acid protein (GFAP)-immunohistochemistry (IHC) staining images of the peripheral nerve tissues according to the second embodiment of the present invention; and

FIGS. 4A to 4F are BLT-1-IHC staining images of the peripheral nerve tissues according to the second embodiment of the present invention.

### DETAILED DESCRIPTION

Hereinafter, various applications of the tissue regeneration membrane for repair and regeneration of peripheral nerve will be described in more details referring to several exemplary embodiments below, while not intended to be limiting. Thus, one skilled in the art can easily ascertain the essential advantages and effects of the present invention and, without departing from the spirit and scope thereof, can make various changes and modifications of the invention to adapt it to various usages and conditions.

As aforementioned, the present invention relates to a tissue regeneration membrane for repair and regeneration of peripheral nerve, which includes a collagen membrane and an anti-inflammatory medicinal composition dispersed therein uniformly, for wrapping an injured peripheral nerve.

Typically, the “peripheral nerve” as discussed hereinafter refers to parts of the nervous tissue except the central nervous tissue. Moreover, the “injured peripheral nerve” as discussed hereinafter refers to the peripheral nerve having at least one disorder of neurapraxia (for example, segmental myelin damage or segmental demyelination), axonotmesis and neurotmesis (for example, at least one damage of endoneurium, perineurium and epineurium, complete disruption of the nerve).

The “repair and regeneration” as discussed hereinafter refers to functional recovery (or repair) of the nerve conduction and the regeneration occurred at the injury site of the peripheral nerve after being wrapped by the tissue regeneration membrane of the present invention.

The “tissue regeneration membrane” as discussed hereinafter refers to the one including a collagen membrane and an anti-inflammatory medicinal composition dispersed therein uniformly. In an embodiment, the collagen membrane is consisted of uncrosslinked collagen that is obtained from any source and any site instead of being limited thereto. The collagen can be purified from various collagen-rich tissues of pigs, cows, chickens or other animals, for example. The collagen membrane excludes any crosslinking agent or chemical additive. In an example, the suitable collagen can be a semi-transparent, dense, flexible, expandable and biodegradable matrix.

In another example, there is no limitation to a thickness of the aforementioned tissue regeneration membrane; however, the thickness of the aforementioned tissue regeneration membrane is preferably close to a diameter of the peripheral nerve. In an embodiment, the collagen membrane can have an average thickness of 0.2 μm to 25 μm, for wrapping an injured peripheral nerve with a desired injured length, in which there is no limitation to the desired injured length; however, the desired injured length can include but be not limited to 5 mm to 50 mm.

In application, the aforementioned injured peripheral nerve can be optionally subjected to a pretreatment, for example, epineurial repair, group fascicular repair or perineurial repair, for repairing the injured site of the peripheral nerve. And then, the tissue regeneration membrane wraps the outside of the injured peripheral nerve.

The term “wrap” as discussed hereinafter is defined as to surround or cover the injured peripheral nerve by the tissue regeneration membrane. It should be mentioned that, the tissue regeneration membrane of the present invention excludes to have a tubular shape (or conduct) of a conventional implant because it is hardly to manipulate the tubular implant in the surgery.

In an embodiment, the thickness of the aforementioned tissue regeneration membrane is close to a diameter of the peripheral nerve, so that a difference defined between an average thickness of the tissue regeneration membrane and a diameter of the injured peripheral nerve is no more than 20 percents. More specifically, in an example, the injured peripheral nerve has a larger diameter and a smaller diameter in the desired injured length, a first difference is defined between the larger diameter and the average thickness, a second difference is defined between the average thickness and the smaller diameter. Based on the larger diameter as 100 percents, the first difference can be no more than 20 percents, preferably 5 percents to 20 percents, and more preferably 5 percents to 10 percents. In an alternative way, based on the smaller diameter as 100 percents, the second difference can be no more than 20 percents, preferably 5 percents to 20 percents, and more preferably 5 percents to 10 percents. In those embodiments, it is only required that the average thickness of the tissue regeneration membrane meets one of the above relationships, either the first difference or the second difference.

It should be mentioned that, if the first difference or the second difference was less than 5 percents, the resultant tissue regeneration membrane would result in poor strength, easy brokenness during following operations and poor isolation from infiltration of peripheral inflammatory cells. In addition, if the first difference or the second difference was more than 20 percents, the resultant tissue regeneration membrane would be hardly to wrap the nerve and have poor permeability.

In those embodiments, the aforementioned tissue regeneration membrane can have a larger thickness and a smaller thickness, in which a third difference is defined between the larger thickness and the smaller thickness, and the third difference is no more than 5 μm, preferably no more than 2 μm, and more preferably no more than 500 nm.

The term “anti-inflammatory medicinal composition” discussed thereinafter typically includes an anti-inflammatory active ingredient and a medicinally acceptable carrier. The aforementioned anti-inflammatory active ingredient can include but be not limited to at least one of cyclooxygenase inhibitor, 5-lipoxygenase (5-LO) inhibitor and leukotriene receptor antagonist.

Examples of the aforementioned cyclooxygenase inhibitor can be acetylsalicylic acid (the trade name of Aspirin, 25 uM), Indomethacin or any combination thereof, for example. Those ingredients have the effect of anti-inflammation of peripheral nerves.

Examples of the aforementioned 5-LO inhibitor can be Zileuton (the trade name such as Zyflo, 25 uM, Sigma-Aldrich), for example. Those ingredients also have the effect of anti-inflammation of peripheral nerves.

Examples of the aforementioned leukotriene receptor antagonist can be Montelukast (the trade name such as Singulair, 500 ng/mL), for example. Those ingredients also have the effect of anti-inflammation of peripheral nerves.

The aforementioned anti-inflammatory active ingredient can be used alone or in combination of two or more anti-inflammatory active ingredients; for example, at least one or two ingredients selected from the group consisting of acetylsalicylic acid, Indomethacin, Zileuton, Montelukast and any combination thereof. In an example, the aforementioned anti-inflammatory active ingredient can be combined with acetylsalicylic acid and Zileuton. In other examples, aforementioned anti-inflammatory active ingredient can be at least three ingredients selected from the group consisting of acetylsalicylic acid, Indomethacin, Zileuton, Montelukast and any combination thereof.

The term “medicinally acceptable carrier” discussed hereinafter refers to a non-active ingredient itself, including carriers, diluents, adjuvants and/or medium, all of which is suitable for administrating the active ingredient to an organism. The medicinally acceptable carrier can be added into the aforementioned composition for improving its treating or storing characteristics. Alternatively, for the purposes of allowing or contributing to form the given dosage unit of such composition, the medicinally acceptable carrier can be also excipients or any material suitably impregnated into the collagen membrane for administration in any convenient manner. The medicinally acceptable carrier should not destroy pharmaceutical activity of those active ingredients, and it also should be nontoxic when the active ingredients is administrated in the sufficient therapeutic dosage.

The aforementioned medicinally acceptable carrier, which can be the ones well known by the skilled person in the field of medical composition of the medical manufacture industry, includes but is not limited to buffering agents, diluents, disintegrants, adhesives, binders, humectants, polymers, lubricants, glidants, substances added for deodorants for obscuring or counteracting bad taste or odor, dyes, fragrances and substances added for improving the appearance of such composition. Examples of the aforementioned medicinally acceptable carrier can include but be not limited to citrate salt buffering agents, phosphate salt buffering agents, acetate salt buffering agents, bicarbonate salt buffering agents, stearic acid, magnesium stearate, magnesium oxide, sodium salts and calcium salts of phosphate and sulfate, magnesium carbonate, talc, gelatin, arabic gum, sodium alginate, pectin, dextrin, mannitol, sorbitol, lactose, sucrose, starch, cellulose materials (for example, esters of cellulose alkanoates and cellulose alkyl esters), low melting point wax, cocoa butter, amino acids, urea, alcohols, ascorbic acid, phospholipids, proteins (for example, serum albumin), ethylenediaminetetraacetic acid (EDTA), dimethyl sulfoxide (DMSO), sodium chloride or other salts, liposomes, glycerol or powder, polymers (for example, polyvinyl pyrrolidone, polyvinyl alcohol and polyethylene glycol) and other medicinally acceptable substances.

In application, a total concentration of the aforementioned anti-inflammatory active ingredient within the tissue regeneration membrane is typically far less than a given concentration of the active ingredient in the oral or injection dose form; however, in the peripheral nerve tissue or the blood, the total concentration of the aforementioned anti-inflammatory active ingredient released from the tissue regeneration membrane is more than the given concentration of the active ingredient in the oral or injection dose form, so that the tissue regeneration membrane can more effectively improve the neuroinflammation. More specifically, in the wrapped peripheral nerve tissue, the total concentration of the aforementioned anti-inflammatory active ingredient released from the tissue regeneration membrane can be 20 ng/mL to 500 ng/mL, for example; however, in the blood, the total concentration of the aforementioned anti-inflammatory active ingredient released from the tissue regeneration membrane can be no more than 20 ng/mL. The concentration of the aforementioned anti-inflammatory active ingredient in the conventionally oral dose form is less in the blood, for example, aspirin (0.832-1.66 mM), Indomethacin (0.16 mM), Zileuton (8.46-25.38 uM), Montelukast (20 ng/mL), all of which is no more than 20 ng/mL. In comparison, the aforementioned anti-inflammatory active ingredient in the tissue regeneration membrane of the present invention is effectively released to the peripheral nerve tissue, so that the concentrations of the anti-inflammatory active ingredient at the site wrapped by the tissue regeneration membrane and surrounding tissues can be both more than 20 ng/mL to 500 ng/mL, for example, aspirin (0.832-1.66 mM), Indomethacin (0.16 mM), Zileuton (8.46-25.38 uM), Montelukast (20 ng/mL), all of which is no more than 20 ng/mL. Thus, the improvement effect provided by the tissue regeneration membrane on the locally wrapped site and the surrounding tissues is far better than the effect provided by the conventionally oral dose form.

Furthermore, according to the evidences of the animal model of the sciatic nerve transection, after the transected sciatic nerve is sutured by neurosurgery and wrapped by the tissue regeneration membrane of the present invention for a desired period, for example, 6 weeks, the infiltration of the inflammatory cells at the injured sciatic nerve can be decreased, the inflammatory signals occurred at the outside and the inside of the injured sciatic nerve, thereby inhibiting the generation of the neuroscar.

Since the thickness of the tissue regeneration membrane for repair and regeneration of peripheral nerve is close to the diameter of the injured peripheral nerve, it can advantageously protect the injured peripheral nerve from the infiltration of surrounding inflammatory cells, and the anti-inflammatory medicinal composition can inhibit neuroinflammation of the injured peripheral nerve, thereby promoting the repair and regeneration of the injured peripheral nerve.

Thereinafter, various applications of the tissue regeneration membrane for repair and regeneration of peripheral nerve will be described in more details referring to several exemplary embodiments below, while not intended to be limiting. Thus, one skilled in the art can easily ascertain the essential characteristics of the present invention and, without departing from the spirit and scope thereof, can make various changes and modifications of the invention to adapt it to various usages and conditions.

### EXAMPLE 1

### Preparation of Tissue Regeneration Membrane

In this EXAMPLE, a commercially available collagen membrane (for example, the product of Sunmax Biotechnology Co., Ltd. made by medical-grade type I atelocollagen having triplex structure and an average molecular weight of 300 kD) was used as a carrier, which appeared semi-transparent, dense, flexible, expandable and biodegradable matrix. The commercially available collagen membrane was cut into pieces of any desired size (for example, each having 2 cm in length, 1.5 cm in width and 0.001 cm in thickness approximately), impregnating in the phosphate buffered saline (PBS; pH7.4) containing various kinds of the anti-inflammatory active ingredients. The anti-inflammatory active ingredient included Zileuton (25 uM, Sigma-Aldrich), aspirin (25 uM), Singulair (500 ng/mL, Merck & CO, Inc., Whitehouse Station, N.J.08889, USA), for example. After impregnated in PBS containing those anti-inflammatory active ingredients for 1 to 2 hours under the room temperature (10° C. to 40° C.), the pieces of the commercially available collagen membrane absorbed completely the anti-inflammatory active ingredients, appeared into slightly white, soft and flexible, and those pieces could be subsequently utilized to wrap the nerve sutured by neurosurgery.

### EXAMPLE 2

### Establishment of Animal Test Model

In the EXAMPLE, rats of the sciatic nerve transection were established as the animal model to evaluate the effect of the tissue regeneration membrane of EXAMPLE 1 for repairing and regenerating the peripheral nerve.

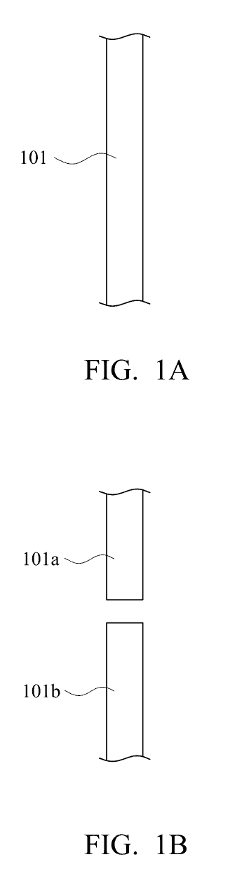

Reference was made to FIGS. 1A to 1D, which were depicted to flow diagrams showing a partial surgical process of establishment of the animal test model according to one embodiment of the present invention. Firstly, as shown in FIG. 1A, Spraque-Dawley (SD) rats (8-week old, 250 to 300 g in body weight) were anesthetized using Zoletil (50 mg/kg intraperitoneally) (Virbac, Carros, France), for example. An approximate 2-cm incision was made from the left sciatic notch to the distal thigh of each rat. The muscle layer (i.e. the biceps femoris muscle) was exposed and the sciatic nerve 101 was freed from the investing fascia. And then, as shown in FIG. 1B, the sciatic nerve 101 was transected to the sciatic nerve 101a and the sciatic nerve 101b at the meddle point between the point of emergence from the spinal cord and the sciatic nerve 101 before the sciatic nerve branches. Following, as shown in FIG. 10, the sciatic nerve 101a and the sciatic nerve 101b were repaired by a surgical suture (9-0 nylon suture) 103 under the light microscope (i.e. Sham control group, Sham).

Later, as shown in FIG. 1D, the sutured site of the injured sciatic nerve was wrapped by the tissue regeneration membrane 105, for example, the collagen membrane (without drugs; i.e. Collagen wrap control group) or the tissue regeneration membrane 105 of EXAMPLE 1 (with drugs; i.e. Collagen+Drug group), fixed by the surgical suture 107, and then observed for 6 weeks. The surgical suture 107 could have the approximately equal width to the surgical suture 103. The rats of all groups of this EXAMPLE were fed with standard laboratory chow and water ad libitum according to the experimental procedures approved by the Institutional Animal Care and Use Committee (IACUC) at National Cheng Kung University (NCKU), Taiwan.

### EXAMPLE 3

### Evaluation of Effect of Tissue Regeneration Membrane for Repairing and Regenerating Peripheral Nerve

### 1. Evaluation of Infiltration of Inflammatory Cells

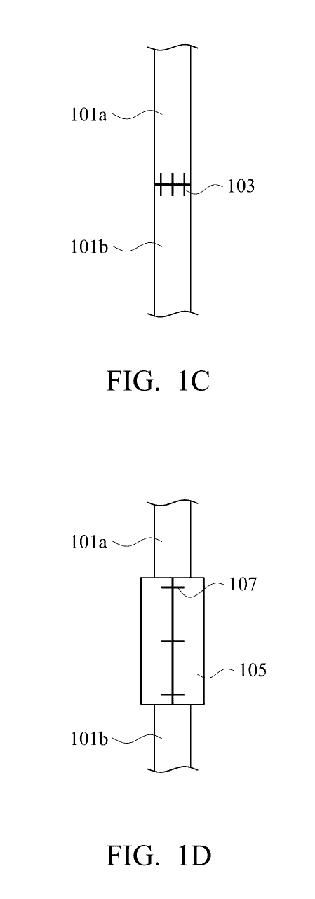

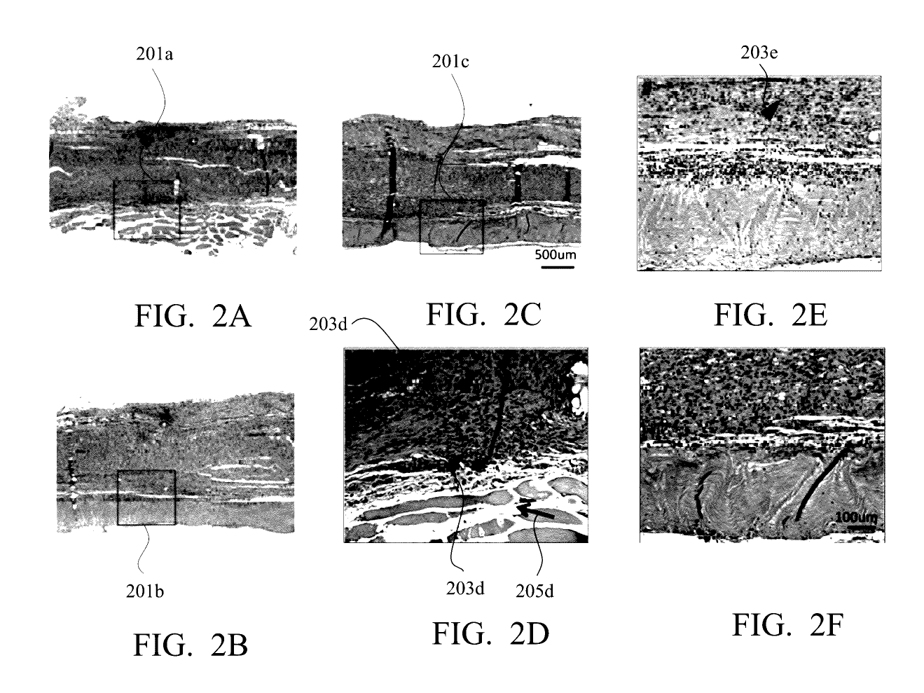

All rats of EXAMPLE 2 were scarified until six weeks after surgery, and the sciatic nerves were harvested and analyzed histologically. At first, the tissues within 0.5 cm around the sutured site of the sciatic nerve segments (about 1 cm in the total length) were dehydrated and fixed by formalin solution, embedded in the paraffin wax blocks, and sectioned transversely into thin sections of each 10 μm in thickness with a microtome. The sections were mounted on glass slides, stained by hematoxylin and eosin (H&E) and shown in FIGS. 2A to 2F. FIGS. 2A and 2D were images depicting the results of “Sham control” groups (Sham). FIGS. 2B and 2E were images depicting the results of “Collagen wrap control” groups. FIGS. 2C and 2F were images depicting the results of “Collagen+Drug” groups (in the case of Zileuton as the active ingredient). FIG. 2D was an image depicting an enlarged diagram of the square 201a of FIG. 2A. FIG. 2E was an image depicting an enlarged diagram of the square 201b of FIG. 2B. FIG. 2F was an image depicting an enlarged diagram of the square 201c of FIG. 2C.

As shown in the results of FIGS. 2A and 2D, severe muscle adhesion (as indicated by the fine arrow 205d in FIG. 2D) was observed at the neurointerface in a low magnification field in the Sham control group; however, there was no adhesion was observed outside the neurointerface in other two Collagen groups (i.e. Collagen wrap control group or the Collagen+Drug group). In a high magnification field, there were a large number of inflammatory cells infiltrated inside or outside the sciatic nerve (as indicated by the bold arrow 203d in FIG. 2D). As shown in the results of FIGS. 2B and 2E, inflammatory cells infiltrated were observed outside the sciatic nerve in a relatively reduced level in the Collagen wrap control group; however, there were still a large number of inflammatory cells infiltrated inside the sciatic nerve (as indicated by the bold arrow 203e in FIG. 2E). As shown in the results of FIGS. 2C and 2F, the Collagen+Drug group, there was a substantial reduction of a large number of inflammatory cells infiltrated inside or outside the nerve. It was evidenced that the tissue regeneration membrane (in the case of Zileuton as the active ingredient) could substantially decrease inflammatory cells infiltrated inside or outside the sciatic nerve segment.

### 2. Evaluation of the Inflammation of the Injured Nerve

For the purpose of further scarring inside and outside the nerve during the nerve regeneration, in this EXAMPLE, immunohistochemistry (IHC) staining performed to detect the expression patterns exhibited by glial fibrillary acid protein (GFAP), for evaluating the neuroscarring level.

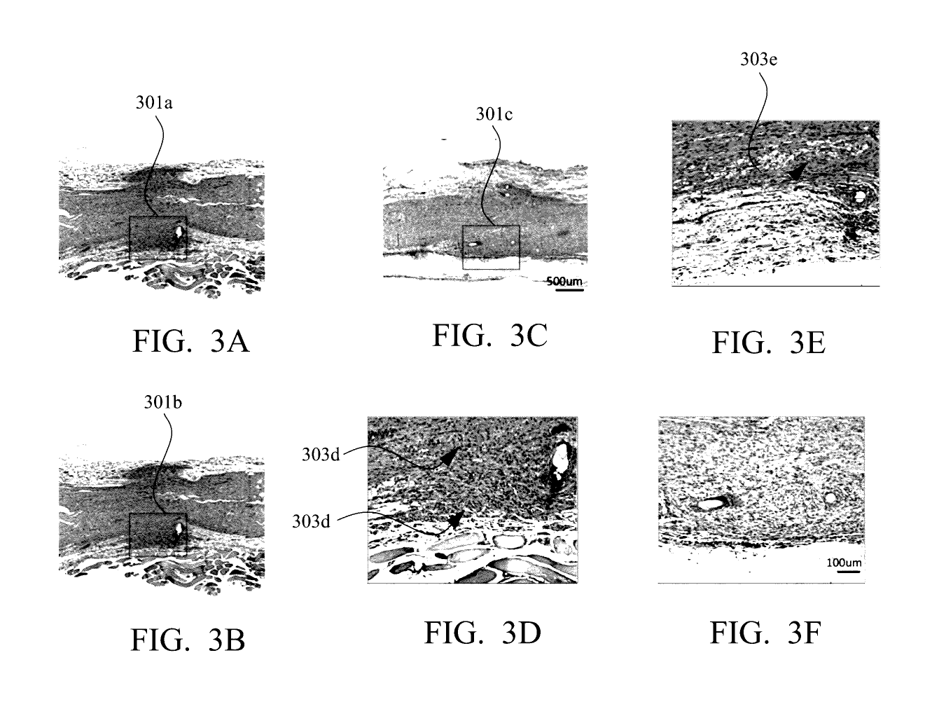

At first, the aforementioned sciatic nerve segments of those groups were embedded in paraffin, sectioned transversely, dewaxed and dehydrated. These sections were treated with rabbit anti-rat glial fibrillary acidic protein (anti-GFAP) monoantibody (dilution of 1:400, Millipore) as a primary antibody, treated with anti-rabbit secondary antibody (dilution of 1:500, Abcam, Cambridge, UK), stained by a chemogen [for example, diaminobenzidine (DAB), Dako, Calif., USA] and resulted in IHC images of the surrounding nerve tissue shown in FIGS. 3A to 3F. FIGS. 3A and 3D were images depicting the results of “Sham control” groups (Sham). FIGS. 3B and 3E were images depicting the results of “Collagen wrap control” groups. FIGS. 3C and 3F were images depicting the results of “Collagen+Drug” groups (in the case of Zileuton as the active ingredient). FIG. 3D was an image depicting an enlarged diagram of the square 301a of FIG. 3A. FIG. 3E was an image depicting an enlarged diagram of the square 301b of FIG. 3B. FIG. 3F was an image depicting an enlarged diagram of the square 301c of FIG. 3C.

As shown in the results of FIGS. 3A and 3D, a number of GFAP signals (as indicated by the bold arrow 303d in FIG. 3D) revealed outside and inside the sutured site of the injured nerve in the Sham control group, indicating that the scar tissue was proliferated and spread out. However, as shown in the results of FIGS. 3B and 3E, GFAP signals (as indicated by the bold arrow 303e in FIG. 3E) were weakened outside the injured nerve in the Collagen wrap control group, indicating that the tissue regeneration of EXAMPLE 1 could substantially have the effect of preventing scar tissue proliferation outside the injured nerve, but it was useless to inhibit the scar tissue proliferation inside the injured nerve. As shown in the results of FIGS. 3C and 3F, only the tissue regeneration membrane of the Collagen+Drug group (in the case of Zileuton as the active ingredient) could effectively inhibit GFAP signals inside and outside the injured nerve, evidencing that the tissue regeneration membrane of EXAMPLE 1 could reduce the undesired neuroscars during the regeneration process of the peripheral nerve.

### 3. Evaluation of Neuroinflammatory Signals

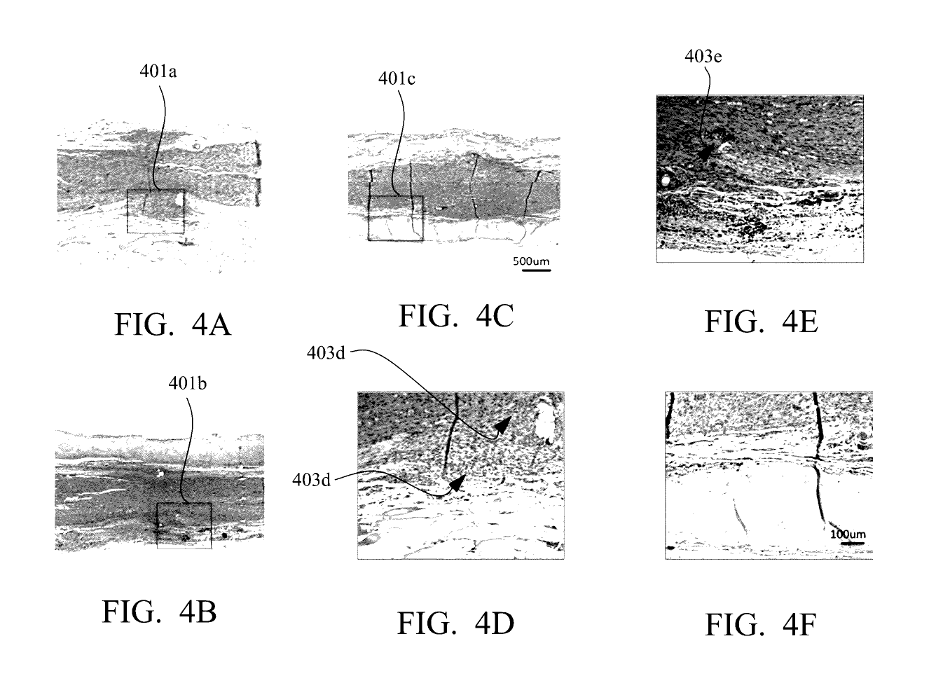

In this EXAMPLE, using the same IHC-staining method as aforementioned, the aforementioned peripheral nerve tissue sections were treated with leukotriene B4 receptor-1 (BLT-1, dilution of 1:100, Cayman) antibody as the primary antibody and resulted in IHC images of the surrounding nerve tissue shown in FIGS. 4A to 4F. FIGS. 4A and 4D were images depicting the results of “Sham control” groups (Sham). FIGS. 4B and 4E were images depicting the results of “Collagen wrap control” groups. FIGS. 4C and 4F were images depicting the results of “Collagen+Drug” groups (in the case of Zileuton as the active ingredient). FIG. 4D was an image depicting an enlarged diagram of the square 401a of FIG. 4A. FIG. 4E was an image depicting an enlarged diagram of the square 401b of FIG. 4B. FIG. 4F was an image depicting an enlarged diagram of the square 401c of FIG. 4C.

As shown in the results of FIGS. 4A and 4D, a number of BLT-1 signals (as indicated by the bold arrow 403d in FIG. 4D) revealed outside and inside the sutured site of the injured nerve in the Sham control group, indicating that the BLT-1 inflammatory signal played an important role in the unwrapped regenerated nerve. However, as shown in the results of FIGS. 4B and 4E, BLT-1 signal (as indicated by the bold arrow 403e in FIG. 3E) could merely provide a very limited inhibition of the infiltration inside the injured nerve in the Collagen wrap control group, even though the Collagen membrane was able to prevent the infiltration outside the injured nerve caused by the BLT-1 signal. As shown in the results of FIGS. 4C and 4F, only the tissue regeneration membrane of the Collagen+Drug group (in the case of Zileuton as the active ingredient) could effectively inhibit in e increase of the signals both inside and outside the injured nerve, evidencing that the tissue regeneration membrane of EXAMPLE 1 could reduce the neuroinflammatory signals during the regeneration process of the peripheral nerve.

In conclusion, the tissue regeneration membrane of EXAMPLE 1 is applied to wrap the sutured peripheral nerve, it can provide significantly therapeutic effects including decreased infiltration of the inflammatory cells, prevention of the neuroscar formation, inhibition of the neuroinflammatory signals, and all therapeutic effects are strongly related to the histological results, evidencing that the tissue regeneration membrane of EXAMPLE 1 can promote the repair and regeneration of the injured peripheral nerve.

Each data of each sample of the aforementioned EXAMPLES was computed as the standard deviation of the mean within triple repeats at each time, all values of which was analyzed by independent t test, and the data referred to statistical significance as p value less than 0.05.

According to the embodiments of the present invention, the aforementioned tissue regeneration membrane for repair and regeneration of the peripheral nerve of the present invention facilitates the repair and regeneration ability of the injured peripheral nerve. It is noted that, the aforementioned EXAMPLES show that the aforementioned tissue regeneration membrane for repair and regeneration of EXAMPLE 1 can prevent infiltration of inflammatory cells inside and outside the injured nerve, inhibit overinflammation of the injured nerve tissues and promote the recovery and the regeneration of injured peripheral nerve, all of which has been evidenced by the animal model of the sciatic nerve transection of EXAMPLE 2. It can be expected that the tissue regeneration membrane can beneficially provide the therapeutic effects of repair and regeneration of the injured peripheral nerves in other cases of the less severe injured nerves (for example, neurapraxia, axonotmesis, less severe neurotmesis and so on).

It is necessarily supplemented that, specific collagen membrane of specific materials and specific sizes, specific anti-inflammatory medicinal composition including specific active ingredients, specific analysis methods or specific apparatuses are exemplified for elucidating the tissue regeneration membrane for repair and regeneration of the peripheral nerve of the present invention. However, as is understood by a person skilled in the art, other collagen membrane of other materials and other sizes, other anti-inflammatory medicinal composition including other active ingredients, other analysis methods or other apparatuses can be also adopted in the tissue regeneration membrane for repair and regeneration of the peripheral nerve of the present invention, rather than being limited thereto.

According to the embodiments of the present invention, the aforementioned tissue regeneration membrane for repair and regeneration of the peripheral nerve of the present invention advantageously includes a collagen membrane having an average thickness very close to a diameter of the injured peripheral nerve and an anti-inflammatory medicinal composition dispersed the collagen membrane uniformly, for wrapping and facilitating regeneration of the injured peripheral nerves.

Although the present invention has been described in considerable detail with reference to certain embodiments thereof, other embodiments are possible. Therefore, the spirit and scope of the appended claims should not be limited to the description of the embodiments contained herein.

What is claimed is:

1. A tissue regeneration membrane which is consisted of uncrosslinked collagen, wherein the tissue regeneration membrane has an average thickness of 0.2 μm to 25 μm, the tissue regeneration membrane wraps an injured peripheral nerve with a desired injured length, the injured peripheral nerve has a larger diameter and a smaller diameter in the desired injured length, a first difference is defined between the larger diameter and the average thickness, a second difference is defined between the average thickness and the smaller diameter, and
wherein the first difference is no more than 20 percents based on the larger diameter as 100 percents, or
the second difference is no more than 20 percents based on the smaller diameter as 100 percents.

2. A tissue regeneration membrane for repair and regeneration of peripheral nerve, comprising a collagen membrane and an anti-inflammatory medicinal composition dispersed therein uniformly, wherein the collagen membrane is consisted of uncrosslinked collagen, and the collagen membrane wraps an injured peripheral nerve with a desired injured length.

3. A tissue regeneration membrane for repair and regeneration of peripheral nerve, comprising:
a collagen membrane, wherein the collagen membrane has an average thickness of 0.2 μm to 25 μm; and
an anti-inflammatory medicinal composition dispersed in the collagen membrane uniformly, and
wherein the tissue regeneration membrane wraps an injured peripheral nerve with a desired injured length, the injured peripheral nerve has a larger diameter and a smaller diameter in the desired injured length, a first difference is defined between the larger diameter and the average thickness, a second difference is defined between the average thickness and the smaller diameter, and
based on the larger diameter as 100 percents, the first difference is no more than 20 percents, or based on the smaller diameter as 100 percents, the second difference is no more than 20 percents.

4. The tissue regeneration membrane for repair and regeneration of peripheral nerve of claim 3, wherein the collagen membrane is consisted of uncrosslinked collagen type I.

5. The tissue regeneration membrane for repair and regeneration of peripheral nerve of claim 3, wherein the first difference is 5 percents to 20 percents based on the larger diameter as 100 percents, or the second difference is 5 percents to 20 percents based on the smaller diameter as 100 percents.

6. The tissue regeneration membrane for repair and regeneration of peripheral nerve of claim 3, wherein the first difference is 5 percents to 10 percents based on the larger diameter as 100 percents, or the second difference is 5 percents to 10 percents based on the smaller diameter as 100 percents.

7. The tissue regeneration membrane for repair and regeneration of peripheral nerve of claim 3, wherein the tissue regeneration membrane has a larger thickness and a smaller thickness, a third difference is defined between the larger thickness and the smaller thickness, and the third difference is no more than 5 μm.

8. The tissue regeneration membrane for repair and regeneration of peripheral nerve of claim 7, wherein the third difference is no more than 2 μm.

9. The tissue regeneration membrane for repair and regeneration of peripheral nerve of claim 7, wherein the third difference is no more than 500 nm.

10. The tissue regeneration membrane for repair and regeneration of peripheral nerve of claim 7, wherein the desired injured length is 5 mm to 50 mm.

11. The tissue regeneration membrane for repair and regeneration of peripheral nerve of claim 3, wherein the anti-inflammatory medicinal composition comprises an anti-inflammatory active ingredient and a medicinally acceptable carrier, and the anti-inflammatory active ingredient comprises at least one of cyclooxygenase inhibitor, 5-lipoxygenase (5-LO) inhibitor and leukotriene receptor antagonist.

12. The tissue regeneration membrane for repair and regeneration of peripheral nerve of claim 11, wherein the anti-inflammatory active ingredient is at least one selected from the group consisting of acetylsalicylic acid, indomethacin, zileuton and montelukast.

13. The tissue regeneration membrane for repair and regeneration of peripheral nerve of claim 11, wherein the anti-inflammatory active ingredient is at least one selected from the group consisting of acetylsalicylic acid, zileuton and montelukast.

14. The tissue regeneration membrane for repair and regeneration of peripheral nerve of claim 11, wherein the anti-inflammatory active ingredient comprises acetylsalicylic acid and zileuton.

15. The tissue regeneration membrane for repair and regeneration of peripheral nerve of claim 11, wherein an amount of the anti-inflammatory active ingredient in the wrapped peripheral nerve tissue is 20 ng/mL to 500 ng/mL.
